(19) United States
(12) Patent Application Publication
(10) Pub. No.: US 2016/0161444 A1
(43) Pub. Date: Jun. 9, 2016

(54) CO SENSOR AND METHOD FOR MANUFACTURING CO SENSOR

(71) Applicant: NAGASAKI UNIVERSITY, Nagasaki (JP)

(72) Inventors: Yasuhiro SHIMIZU, Nagasaki (JP); Takeo HYODO, Nagasaki (JP); Hirotaka TAKEDA, Nagasaki (JP)

(73) Assignee: NAGASAKI UNIVERSITY, Nagasaki (JP)

(21) Appl. No.: 14/902,187

(22) PCT Filed: Jun. 26, 2014
(86) PCT No.: PCT/JP2014/066965

§ 371 Date: Dec. 30, 2015

(30) Foreign Application Priority Data
Jul. 2, 2013 (JP) 2013-138877

(51) Int. Cl. G01N 27/407; G01N 33/00

(52) U.S. Cl. CPC G01N 27/4074; G01N 33/004; G01N 27/4075

(57) ABSTRACT

A CO sensor 1 is provided with a sensing electrode 3 and a reference electrode 4 on a surface of a solid electrolyte substrate 2. The sensing electrode 3 is composed of Pt, to which Bi₂O₃ is added and the reference electrode 4 is composed of Pt. The sensing electrode 3 has CO selectivity by which CO gas concentration can be sensed and responds rapidly at room temperature.

### TECHNICAL FIELD

The present invention relates to a CO sensor (a carbon monoxide gas sensing sensor: the same shall apply hereinafter) and it especially relates to a CO sensor having a high CO selectivity by using a solid electrolyte and a method for manufacturing the CO sensor.

### BACKGROUND

A constant potential electrolytic gas sensor, a semiconductor type gas sensor, a catalytic combustion type gas sensor and the like are known as the CO sensor. However, since they respond to reducing gas (inflammable gas) indiscriminately in principle, they have a characteristic of sensing H₂ (hydrogen gas: the same shall apply hereinafter) and the like other than CO (carbon monoxide gas: the same shall apply hereinafter). In other words, they have a defect of poor CO selectivity.

A non-patent document 1 describes that a short-circuit current type CO sensor, which uses polybenzimidazole as an electrolyte film and is combined with a Pt supported carbon electrode, has CO selectivity. However, the disclosed data shows that the CO selectivity does not meet any practical requirements and the CO sensor itself requires high temperature operation at 200° C.

The non-patent document 1 further describes that a short-circuit current type CO sensor, which is combined with a cation conductive polymer electrolyte film and a Pt supported carbon electrode, is capable of operating at relatively low temperature (80° C.). However, since it is necessary to operate in a moist atmosphere, it is required to prepare a reservoir which always supplies water, other than a sensor device. Thus, the sensor cannot be down-sized.

In order to eliminate such a water supply, a CO sensor using solid electrolyte described in a patent document 1 (Japanese Patent Publication No. 2002-310983) has been known. In the patent document 1, it is a feature that a pair of electrodes is mounted on a solid electrolyte substrate, such as zirconia and the like, one of the electrodes is coated with CO oxidation catalyst, but the other one of the electrodes is not coated with the catalyst. When CO is generated in the atmosphere, one of the electrodes oxidizes CO, leading to a decrease in oxygen partial pressure. This is detected from an electro motive force of the zirconia. However, the solid electrolyte substrate should be heated to 400-500° C. by using a heater in this CO sensor. And the sensitivity is low because the oxygen partial pressure changes slightly by oxidizing an extremely small amount of CO.

Alternatively, a patent document 2 (Japanese Patent Publication No. 2006-184252) discloses a gas sensor that uses NASICON (for example, Na₃Zr₂Si₂PO₁₂), which is confirmed to have ion conductive property required for the sensor response at low temperature, as the solid electrolyte,

In the patent document 2, a sensing electrode consisting of a gas sensing layer and a charge charge collection layer, and a reference electrode to oppose the sensing electrode are provided on the solid electrolyte substrate. Gold (Au) is used for the electrode material of the charge collection layer and the reference electrode, and the gas sensing layer covering the charge collection layer is a combination of two kinds of metal oxides having different electric resistance values.

The patent document 2 recites Japanese Patent Publication No. H11-271270 as a public known example. This document shows that there are defects of strength poverty of electrodes and poor sensing stability caused by using the metal oxide and metal oxide salt as the gas sensing layer, wherein. composite salt is produced during the gas sensing.

The patent document 2 resolves such defects and an object of the patent document 2 is to accomplish the sufficient electrode strength and sensing stability. A technical means adopted therefor is to use two kinds of metal oxides as the as sensing layer. Firstly, this accomplishes the sufficient electrode strength. This is because mechanical strength of the second metal oxide is high (see paragraph [0010]) so that the mechanical strength can be accomplished and a breakage of the electrode can be prevented by using the second metal oxide.

Secondly, it is difficult for the second metal oxide to form the composite salt with the metal oxide salt, and therefore it is difficult for the first metal oxide to form the composite salt with the first metal oxide. As a result, by using the two kinds of the metal oxides and the metal oxide salt, formation of the composite salt can be suppressed in the gas atmosphere, thereby accomplishing the sensing stability (see paragraph [0011]).

Therefore, the patent document 2 accomplishes the desired object by coating the charge collection layer with the gas sensing layer to make two-layer configuration, configuring the gas sensing layer with two kinds of the metal oxides and the metal oxide salt and using the second metal oxide which gives the strength and the sensing stability.

Further, in the patent document 2, a target gas is sensed by a variation of the electro motive force between the sensing electrode and the reference electrode before and after the contact between the gas to be sensed and the sensing electrode. The disclosed embodiment thereof is a CO₂ sensor (a carbon dioxide gas sensing sensor). The patent document 2 describes its potential as a CO sensor but there is no description of concrete configuration and composition for the CO sensor (see paragraph [0020]).

### DOCUMENT FOR PRIOR ART

### Patent Document

Patent Document 1: Japanese Patent Publication No. 2002-310983

Patent Document 2: Japanese Patent Publication No. 2006-184252

### Non-patent Document

Non-patent Document 1: Journal of The Electrochemical Society, 158 (3) J71-J75 (2011)

### SUMMARY OF THE INVENTION

### Problems to be Solved by the Invention

In such conventional known CO sensors, CO selectivity is poor, sensitivity to CO is not enough and its performance is restricted by environmental temperature. In addition, downsizing is difficult because configuration elements other than the sensor device are required.

Accordingly, the present invention solves such conventional problems and provides a CO sensor which has high CO selectivity. In addition, the present invention provides a CO sensor which can work at room temperature and which can have a simple configuration without water supply, etc. and can be down-sized.

### Means for Solving the Problems

A CO sensor according to the present invention claimed in Claim 1 is characterized in that a sensing electrode and a reference electrode are mounted on a solid electrolyte substrate, Pt to which metal oxide is added is used for the sensing electrode, Pt is used for the reference electrode, and the sensing electrode has a gas sensing function and a charge collection function.

The CO sensor according to the present invention claimed in Claim 2 is characterized in that in the solid electrolyte configuring the substrate, NASICON that is as an ion conductive member is used and the metal oxide added to the sensing electrode is Bi₂O₃.

The CO sensor according to the present invention claimed in Claim 3 is characterized in that an additive amount of the Bi₂O₃ is 0.1 mass % or more, preferably 1 mass % or more but 30 mass % or less.

A method for manufacturing a CO sensor according to the present invention claimed in Claim 4 is characterized in that; in the CO sensor containing a sensing electrode and a reference electrode mounted on a solid electrolyte substrate, the sensing electrode having a gas sensing function and a charge collection function; a method of manufacturing the CO sensor, comprising the steps of using a mixed paste for the sensing electrode, the mixed paste being formed by mixing metal oxide with a Pt paste, printing the mixed paste on the solid electrolyte substrate; and forming the CO sensor by calcinating at a predetermined temperature after printing the Pt paste.

### Effects of the Invention

In this invention, the CO sensor comprises the sensor substrate, the sensing electrode and the reference electrode, wherein the electrodes are formed on the substrate. The solid electrolyte is used for the substrate. Each of the sensing and reference electrodes is a single layer configuration. Pt is especially used as the metal material of the sensing electrode and the reference electrode. Pt to which the metal oxide is added (included) is particularly used for the sensing electrode. The sensing electrode itself has the function of selective sensing of CO gas and the function of collecting the charge generated during the gas sensing.

Thus, the desired object can be accomplished wherein CO selectivity is improved, any heating means is not needed, sensing at normal temperature is possible and downsizing is possible. In other words, according to the present invention, it is possible to accomplish a CO sensor having high CO selectivity, in which the solid electrolyte substrate is used, the sensing electrode is made of Pt to which the metal oxide is added and the reference electrode is made of Pt, wherein both electrodes are formed on the substrate. In addition, according to the invention, it is also possible to accomplish the CO sensor device that is capable of sensing the gas at room temperature.

Of course, the invention is characterized in that it is possible to accomplish the CO sensor Which can be down-sized since the sensor device can be configured without any necessary of accompanying sensing members, etc. for supplying water during the sensing operation.

In the method of manufacturing the CO sensor according to the present invention, after the pair of electrodes is formed on the solid electrolyte substrate by screen printing, they are only calcinated. Therefore, the down-sized CO sensor can be manufactured easily.

### BRIEF DESCRIPTION OF DRAWINGS

FIG. 1A is a structure plane view of a C sensor showing a configuration example thereof according to the present invention.

FIG. 1B is a cross sectional view of the CO sensor showing a configuration example thereof according to the present invention.

FIG. 2A is a secondary electron microscope photograph of a surface of a sensing electrode.

FIG. 2B is a secondary electron microscope photograph of a surface of a reference electrode.

FIG. 3 is a characteristics graph showing a response transient to CO and H₂ when the additive amount of Bi₂O₃ used as the metal oxide is 0.01 mass %.

FIG. 4 is a characteristics graph showing a response transient to CO and H₂ when additive amount of Bi₂O₃ used as the metal oxide is 0.1 mass %.

FIG. 5 is a characteristics graph showing a response transient to CO and H₂ when the additive amount of Bi₂O₃ used as the metal oxide is 1 mass %.

FIG. 6 is a characteristics graph showing a response transient to CO and H₂ when the additive amount of Bi₂O₃ used as the metal oxide is 10 mass %.

FIG. 7 is a characteristics graph showing a response transient to CO and H₂ when the additive amount of Bi₂O₃ used as the metal oxide is 30 mass %.

FIG. 8 is a characteristics graph showing responses to CO when the additive amount of Bi₂O₃ used as the metal oxide is varied.

FIG. 9 is a characteristics graph showing responses to H₂ when the additive amount of Bi₂O₃ used as the metal oxide is varied.

FIG. 10 is a characteristics graph showing a response transient to CO at 100° C. of a comparison example using Au as the electrode materials and the embodiment using Pt as the electrode materials.

### EMBODIMENT FOR CARRYING OUT THE INVENTION

### Embodiment

The following will describe the most suitable embodiment for carrying out the present invention.

(1) Configuration of CO Sensor

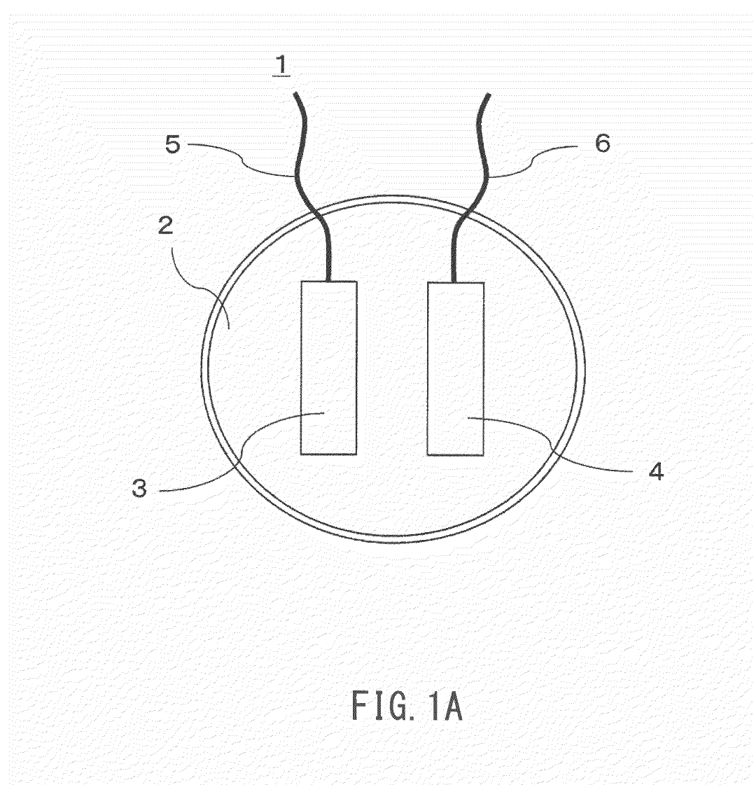

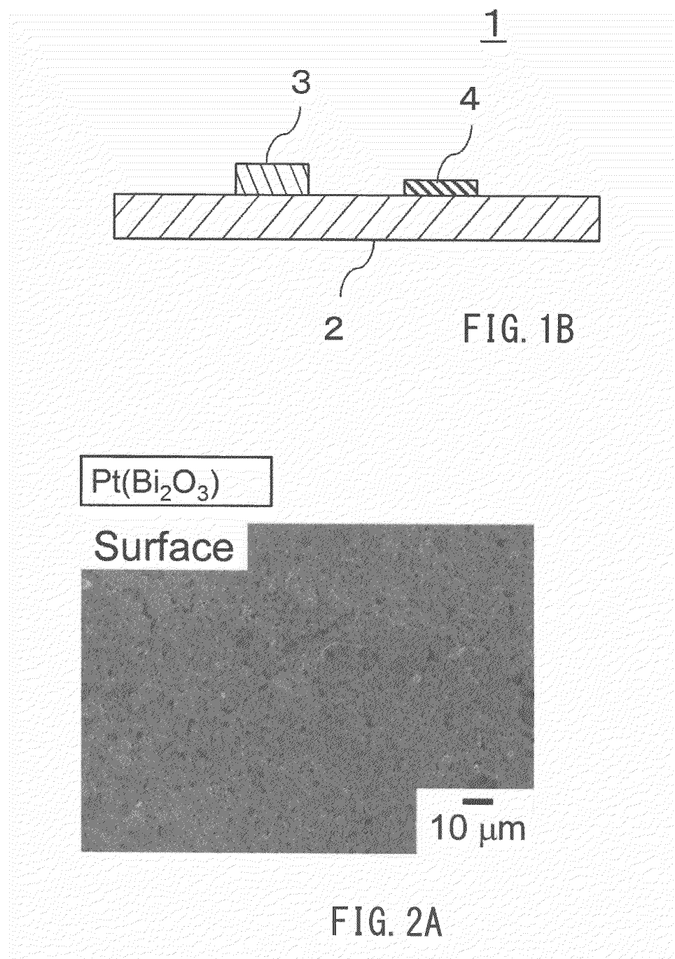

FIGS. 1A and 1B show a configuration example of a CO sensor 1 according to the present invention. As shown in FIG. 1A, the CO sensor 1 contains a substrate 2, a sensing electrode 3 and a reference electrode 4, wherein both electrodes are formed on the substrate 2 approximately in center.

The substrate 2 is a substrate made of a solid electrolyte, and NASICON (Na Super Ionic Conductor) of an ion conductive material is used for the solid electrolyte. Na₃Zr₂Si₂PO₁₂ and the like are preferable for NASICON.

As also illustrated in FIG. 1B, the solid electrolyte substrate 2 is a discotic substrate, dimensions of which are 8 mm in diameter and 0.7 mm in thickness of in this example. In the sensing electrode 3 for sensing CO, platinum (Pt) is used for an electrode material and a predetermined amount of metal oxide is added to the electrode material Pt. Bi₂O₃ is used for the metal oxide. The sensing electrode 3 is a square shaped thick film, dimensions of which are 1 mm in width, 4 mm in length and 30 μm in thickness, approximately. The sensing electrode 3 itself has a function of sensing CO gas and a function of collecting charges generated during the gas sensing, which will be described hereinafter,

For the metal material of the reference electrode 4, Pt is used similarly to the sensing electrode. The reference electrode 4 is a square shaped thick film, dimensions of which are 1 mm in width, 4 mm in length and 20 μm thickness, approximately, as shown in the drawing. A Pt thin wire of 0.1 mm in diameter is used for both of a wire 5 for the sensing electrode 3 and a wire 6 for the reference electrode 4. When designing a circumference of the sensor, the shapes of the sensing electrode 3 and the reference electrode 4 can be optimized in many shapes to improve the CO diffusivity and the CO reactivity at the surface of the electrode (one example will be described hereinafter).

(2) Method for Manufacturing CO Sensor

The following will describe an example of a method for manufacturing the CO sensor 1 according to the present invention. First, a Pt/Bi₂O₃ paste, which is a mixture of Pt and Bi₂O₃, is prepared for the sensing electrode 3 on the sold electrolyte substrate 2 having a predetermined shape. A Pt paste for the reference electrode 4 is similarly prepared. These pastes are sequentially applied by screen printing. Then, the wire 5 is attached to one side of the sensing electrode 3 and the wire 6 is attached to one side of the reference electrode 4. After that, the sensing electrode 3 and the reference electrode 4 are calcinated at 700° C. for 30 minutes to form the CO sensor 1. In this manner, each of the sensing electrode 3 and reference electrode 4 is a single layer configuration.

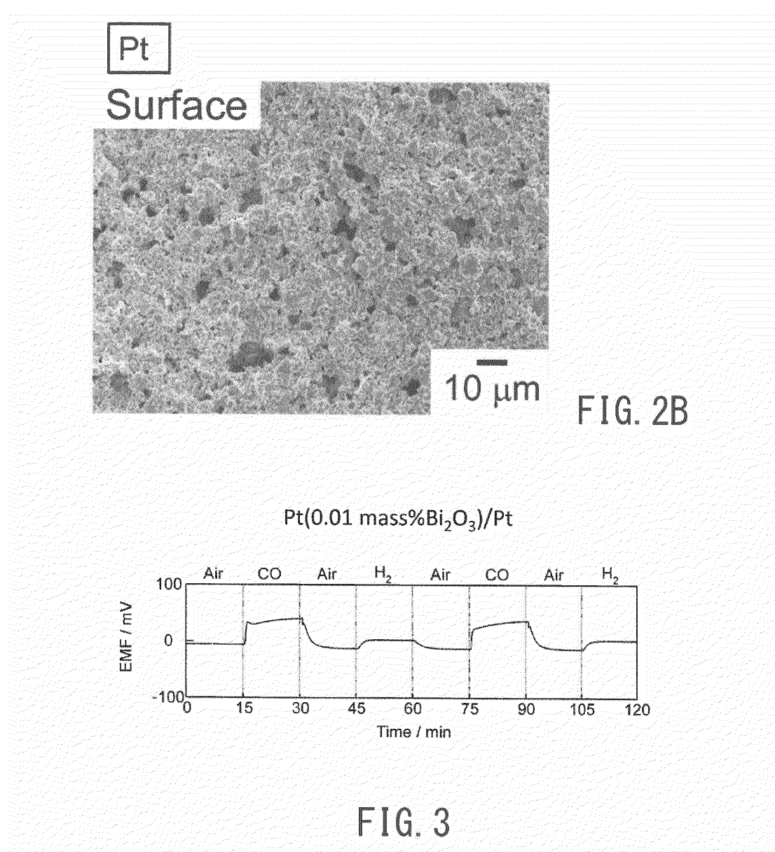

The paste for the sensing electrode 3 is obtained by adding only an amount corresponding to predetermined mass % (about 1-30 mass %) of Bi₂O₃ having a specific surface area of 2.3 m²/g to the Pt paste and by mixing them in a mortar. FIG. 2A and FIG. 2B show examples of electron microscope photographs showing the surfaces of the calcinated sensing electrode 3 and the calcinated reference electrode 4. FIG. 2A is the photograph showing the surface of the sensing electrode 3. In this case, the additive amount of Bi₂O₃ is 15 mass %. FIG. 2B is the photograph showing the surface of the calcinated reference electrode 4. When the both electrodes are compared, a feature is seen, that the surface of the sensing electrode 3 is more dense than that of the reference electrode 4 since Bi₂O₃ is added to the sensing electrode 3.

(3) Measuring Method

A voltmeter (not shown) was connected to the wire 5 and the wire 6 to measure an electro motive force (difference) of the sensing electrode 3 in relation to the reference electrode 4. The electro motive force will be abbreviated as EMF hereinafter. The CO gas having the gas concentration of 300 ppm was used as sample gas under test and the H₂ gas having the gas concentration of 300 ppm was used for verifying the selectivity. The measurement was executed under an air environment at measurement temperatures of 25° C. (normal room temperature), 100° C. and 300° C.

FIG. 3 through FIG. 7 are characteristic graphs each showing response transient of the CO sensor 1 to CO and H₂ at 25° C. FIG. 3 is the characteristic graph when the additive amount of Bi₂O₃ is 0.01 mass %. In the drawing, a horizontal axis represents a time (minute) and a vertical axis represents EMF (mV). In FIG. 3,

(3a) During the first 15 minutes, the atmosphere during sensing is air and the EMF is substantially zero,
(3b) During the next 15 minutes (the elapsed time is from 15 minutes to 30 minutes), the CO gas having its concentration of 300 ppm is blown to the CO sensor 1 at a flow rate of about 100 ml/min. At this time, the EMF rises quickly to about 32 mV and then gradually increases toward about 40 mV.
(3c) During the next 15 minutes (the elapsed time is from 30 minutes to 45 minutes), the atmosphere is returned to the air and then the EMF returns to 0 (mV).
(3d) During the next 15 minutes (the elapsed time is from 45 minutes 60 minutes), the H₂ gas having its concentration of 300 ppm is blown to the CO sensor 1 at a flow rate of about 100 ml/min. At this time, the EMF rises only to about 2 mV.

Therefore, in the example of detection characteristic when the additive amount of Bi₂O₃ is 0.01 mass %, a certain level of the CO selectivity can be confirmed. After the elapsed time of 60 minutes, the sequence of air-CO-air-H₂ . . . is repeated and the similar trend is shown.

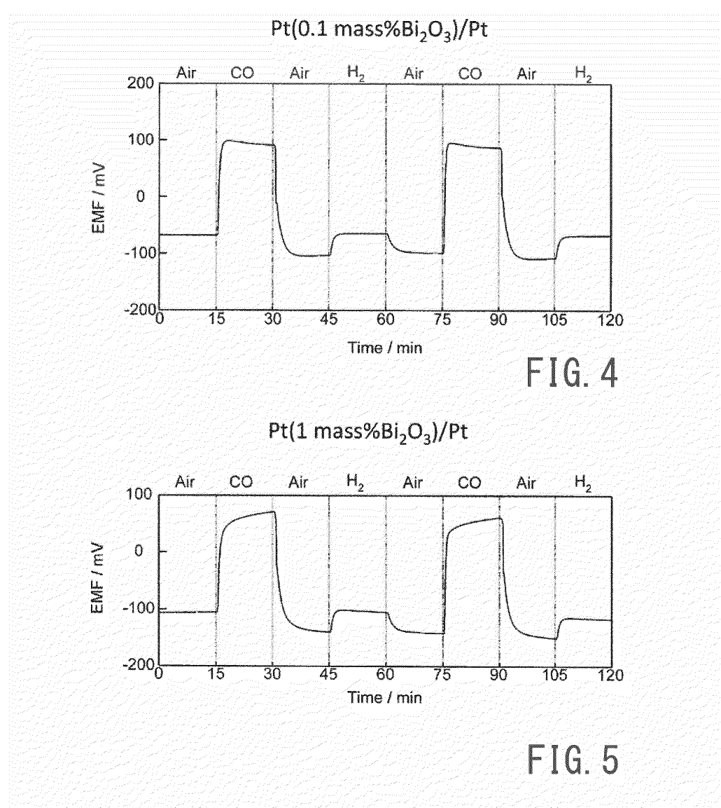

FIG. 4 is the characteristic graph when the additive amount. of Bi₂O₃ is 0.1 mass %. In the drawing,

(4a) In the initial air holding, an initial value of e EMF is about −68 mV.
(4b) During the next 15 minutes (the elapsed time is from 15 minutes to 30 minutes), when the CO gas having its concentration of 300 ppm is blown to the CO sensor 1 at the flow rate of about 100 ml/mm, the EMF rises quickly to about 98 mV and then gradually decreases toward about 91 mV.
(4c) During the next 15 minutes (the elapsed time is from 30 minutes to 45 minutes), the atmosphere is returned to the air, the EMF returns to about −103 mV. A reason why the EMF is lower than the initial value may be based on a CO history effect.
(4dl) During the next 15 minutes (the elapsed time is from 45 minutes 60 minutes), the H₂ gas having its concentration of 300 ppm is blown to the CO sensor 1 at the flow rate of about 100 ml/min. At this time, the EMF rises to about −65 mV.

Therefore, in the example of detection characteristic when the additive amount of Bi₂O₃ is 0.1 mass %, the significant CO selectivity can be confirmed. After the elapsed time of 60 minutes, the sequence of air-CO-air-H₂ . . . is repeated and the similar trend is shown.

FIG. 5 is the characteristic graph when the additive amount of Bf₂O₃ is 1 mass %. In the drawing,

(5a) In the initial air atmosphere, the EMF is about −105 mV.
(5b) During the next 15 minutes (the elapsed time is from 15 minutes to 30 minutes), when the CO gas having its concentration of 300 ppm is blown to the CO sensor 1 at the flow rate of about 100 ml/min, the EMF rises quickly to about 60 mV and then gradually increases toward about 70 mV.
(5c) During the next 15 minutes (the elapsed time is from 30 minutes to 45 minutes), the atmosphere is returned to the air and then the EMF returns to about −140 mV.
(5d) During the next 15 minutes (the elapsed time is from 45 minutes 60 minutes), the H₂ gas having its concentration of 300 ppm is blown to the CO sensor 1 at the flow rate of about 100 ml/min. At this time, the EMF rises to about −105 mV.

Therefore, in the example of detection characteristic, when the additive amount of Bi₂O₃ is 1 mass %, the significant CO selectivity can be confirmed. After the elapsed time of 60 minutes, the sequence of air-CO-air-H₂ . . . is repeated and the similar trend is shown.

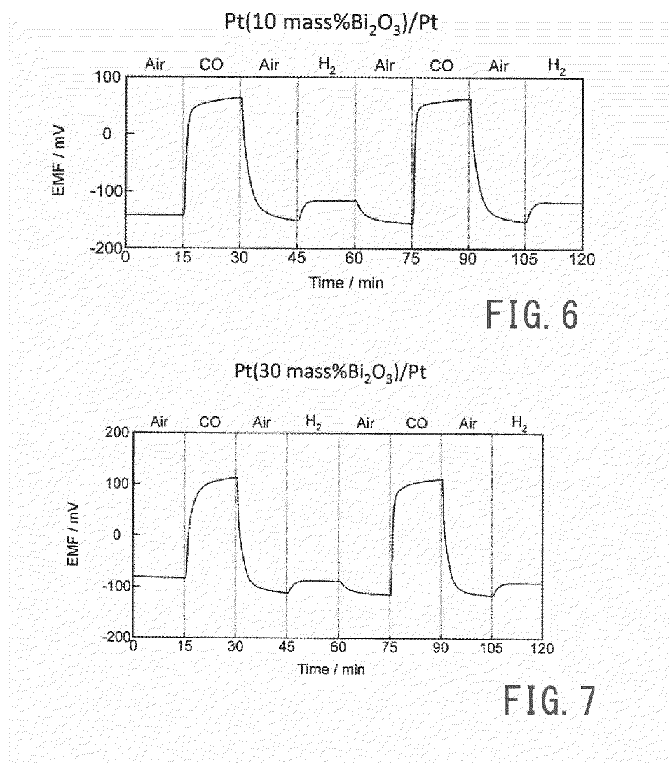

FIG. 6 is the characteristic graph when the additive amount of Bi₂O₃ is 10 mass %. In the drawing,

(6a) In the initial air atmosphere, the EMF is about −142 mV.
(6b) During the next 15 minutes (the elapsed time is from 15 minutes to 30 minutes), when the CO gas having its concentration of 300 ppm is blown to the CO sensor 1 at the flow rate of about 100 ml/min, the EMF rises quickly to about 55 mV and then gradually increases toward about 64 mV.
(6c) During the next 15 minutes (the elapsed time is from 30 minutes to 45 minutes), the atmosphere is returned to the air and then the EMF returns to about −150 mV.
(6d) During the next 15 minutes (the elapsed time is from 45 minutes 60 minutes), the H₂ gas having its concentration of 300 ppm is blown to the CO sensor 1 at the flow rate of about 100 ml/min. At this time, the EMF rises to about −117 mV.

Therefore, in the example of detection characteristic when the additive amount of Bi₂O₃ is 10 mass %, the significant CO selectivity can be confirmed. After the elapsed time of 60 minutes, the sequence of air-CO-air-H₂ . . . is repeated and the similar trend is shown.

FIG. 7 is the characteristic graph when the additive amount of Bi₂O₃ is 30 mass %. In the drawing,

(7a) In the initial air atmosphere, the EMF is about −84 mV.
(7b) During the next 15 minutes (the elapsed time is from 15 minutes to 30 minutes), when the CO gas having its concentration of 300 ppm is blown to the CO sensor 1 at the flow rate of about 100 ml/min, the EMF rises quickly to about 100 mV and then gradually increases toward about 112 mV.
(7c) During the next 15 minutes (the elapsed time is from 30 minutes to 45 minutes), the atmosphere is returned to the air and then the EMF returns to about −111 mV.
(7d) During the next 15 minutes (the elapsed time is from 45 minutes 60 minutes), the H₂ having its concentration of 300 ppm is blown to the CO sensor 1 at the flow rate of about 100 ml/min. At this time, the EMF rises to about −89 mV.

Therefore, in the example of detection characteristic when the additive amount of Bi₂O₃ is 30 mass %, the significant CO selectivity can he confirmed. After the elapsed time of 60 minutes, the sequence of air-CO-air-H₂ . . . is repeated and the similar trend is shown. As shown in the experimental examples above, CO can be selectively sensed by setting the detection level (mV) appropriately.

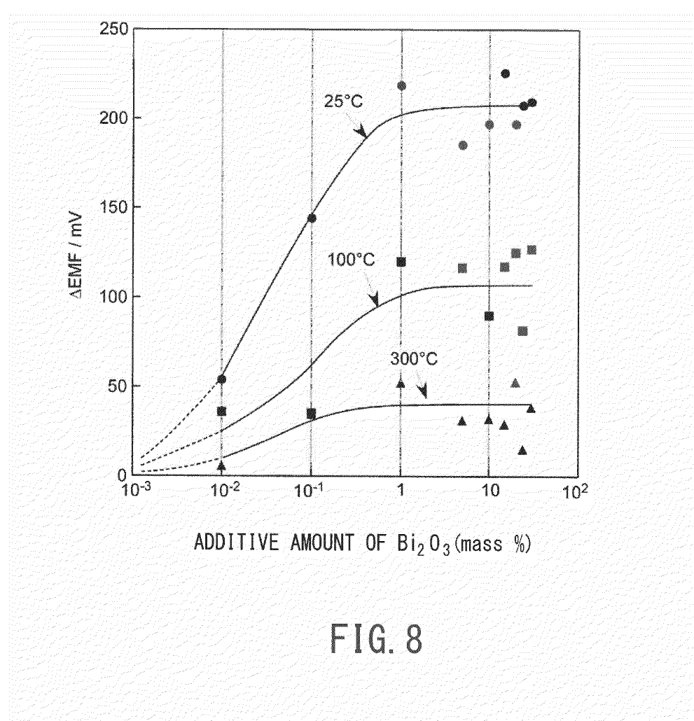

FIG. 8 is a characteristics graph showing responses to CO when the additive amount of Bi₂O₃ and the atmosphere temperature during sensing are varied respectively. A horizontal axis represents the additive amount (mass %) of Bi₂O₃ in a logarithmic scale where a variation of the additive amount of Bi₂O₃ is from 0.01 mass % to 30 mass %. A vertical axis represents ΔEMF (mV) which is the difference between the EMF in CO and the EMF in the air. In detail, in the characteristic graphs shown in FIG. 3 through FIG. 7, ΔEMF is the difference between the EMF in the air just before the second CO blowing and the maximum EMF at the rising just after the CO blowing. In the drawing, black circles represent measurement values at 25° C., black squares represent measurement values at 100° C. and black triangles represent measurement values at 300° C.

Referring to the measurement values at 25° C., ΔEMF is about 50 mV at the Bi₂O₃ additive amount of 0.01 mass %, is about 140 mV at the Bi₂O₃ additive amount of 0.1 mass % and is about 200 mV at the Bi₂O₃ additive amount of 1 mass % or more. Therefore, for the Bi₂O₃ additive amount, 0.1 mass or more is preferable and 1 mass % is more preferable.

If the Bi₂O₃ additive amount is increased, when the Bi₂O₃ is mixed with the Pt paste to form the mixed paste, problems to lower the mechanical strength and the paste viscosity may occur. Therefore, in the manufacturing process for manufacturing the sensing electrode by screen-printing the paste and calcinating it, 30 mass % or less is preferable for the Bi₂O₃ additive amount in order to avoid degradation of the thixotropy.

On the other hand, when looking at the values measured at 100° C., about 100 mV is obtained for the Bi₂O₃ additive amount of 1 mass % or more, so sensing characteristic is sufficient in the temperature range from the normal room temperature (25° C.) to 100° C.

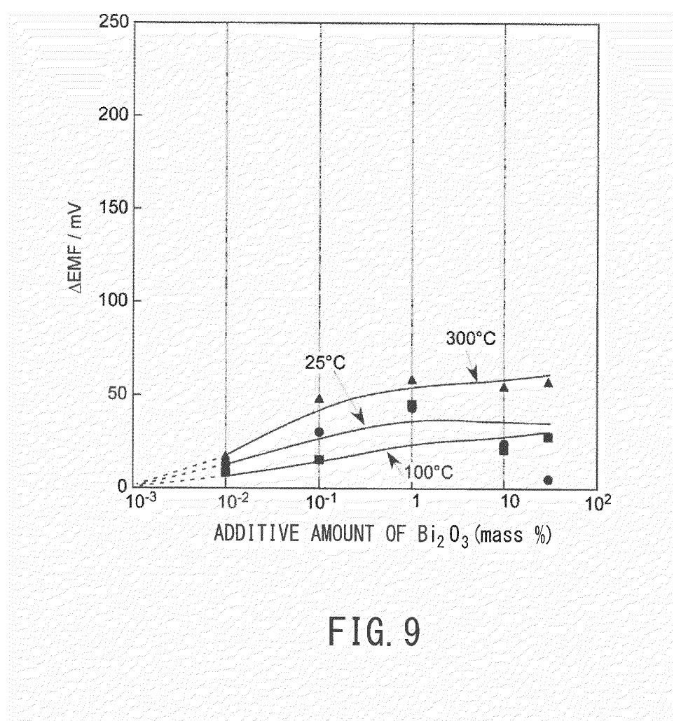

FIG. 9 is a characteristics graph showing responses to H₂ when the additive amount of Bi₂O₃ and the atmosphere temperature during sensing are varied respectively. The drawing shows 30 mV or less at 25° C. and 25 my or less at 100° C. From the result above, it is recognized that the CO sensor 1 of the present embodiments has sufficient selectivity against H₂, in addition to sufficient sensitivity to CO in the temperature atmosphere at normal temperature or more.

In this embodiment, the reaction of the CO sensing may be considered as follows. When CO is adsorbed to the sensing electrode 3, the following reaction occurs in accordance with a high oxidative activity effect of Bi₂O₃.

CO+O²⁻→CO₂+2e⁻ (1)

Then, CO₂ and charges are generated.

Next, they react to ion in the solid electrolyte and the ion activity changes. In this embodiment, Na⁺ of NASICON is subjected to the following reaction.

2Na⁺+CO₂+½O₂+2e⁻→Na₂CO₃ (2)

Then, the Na⁺ activity changes and a potential difference occurs with respect to the reference electrode 4.

As seen in this reaction, the sensing electrode 3 is an electrode having the function of sensing the CO gas and the function of collecting the charges.

In other words, when CO is adsorbed, the charges 2e⁻ are produced as shown by the equation (1) in accordance with the oxidative activity effect of Bi₂O₃. These charges react to the sodium ion Na⁺ in the substrate 2 and the ion activity of the sodium ion changes as shown by the equation (2). That produces the potential difference between the sensing electrode 3 and the reference electrode 4. This potential difference is sensed to represent the variation of the CO concentration.

On the other hand, since such reaction is hard to be occurred in the case where the gas under test is H₂, it may be considered that the selectivity to CO occurs.

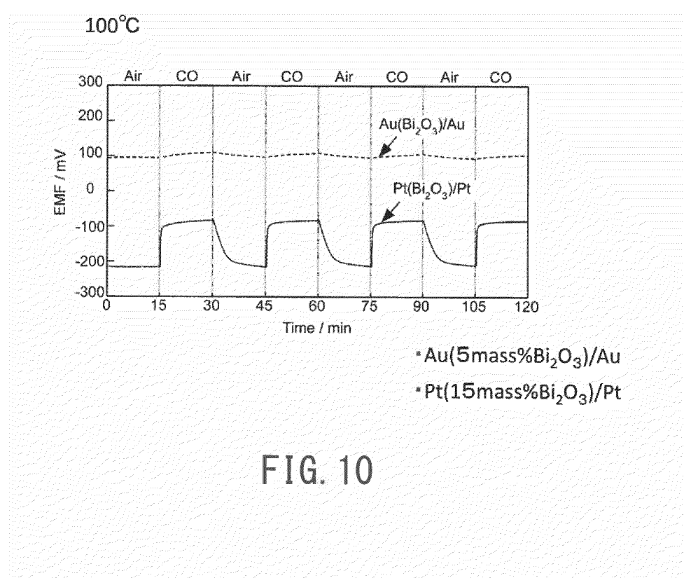

A characteristic comparison between the embodiment of the present invention and a comparison example will be explained by reference to FIG. 10. The comparison example is a CO sensor wherein the sensing electrode 3 is a calcinated Au paste to which Bi₂O₃ of 5 mass % is added and the reference electrode 4 is a calcinated Au paste.

FIG. 10 is a characteristic graph showing response transients to CO at 100° C. wherein a dotted line represents the comparison example and a solid line represents the embodiment (additive amount of Bi₂O₃ is 15 mass %). In FIG. 10, the sequence of the air→CO is repeated every 15 minutes and the EMF is measured.

As for the CO sensor of the embodiment (solid line), there is a difference of about 100 mV between the air and CO. However, the CO sensor used as the comparison example (dotted line) hardly reacts to CO. In the embodiment at 100° C. (black square) shown in FIG. 8, ΔEMF is about 100 mV when the additive amount of Bi₂O₃ is 5 mass %.

A reason of this difference originating from the electrode metal material is thought that the oxidative activity of Pt is higher than that of Au, thereby reinforcing the CO oxidative activity effect of Bi₂O₃.

Therefore, it can be said that a combination of the usage of Pt as the electrode material and the usage of Bi₂O₃ as the metal oxide is preferable to the CO sensor.

(Modification)

In FIG. 1A and FIG. 1B illustrating the example of the embodiment 1, the CO sensor 1 has configured such that the strip shaped sensing electrode 3 and reference electrode 4 are arranged on the circular shaped solid electrolyte substrate 2, wherein the electrodes are in parallel. As described hereinbefore, the shapes of the substrate 2, the sensing electrode 3, the reference electrode 4 and the like can be optimized to many kinds of shapes in order to improve the diffusivity of CO and the CO reactivity on the electrode surface. The following will describe an example of the optimized examples (modification examples) of the electrode shape and the like.

(1) In the case of a disk shaped solid electrolyte substrate 2, a configuration is that an annular shaped (doughnut shaped) sensing electrode 3 is provided on the substrate and a disk shaped reference electrode 4 is provided within it.
(2) In the case of a square plate shaped solid electrolyte substrate 2, a configuration is that a square shaped sensing electrode 3 is provided on the substrate and a square or disk shaped reference electrode 4 is provided within it.
(3) A configuration is that the sensing electrode 3 and the reference electrode 4 having the shapes of the embodiment are considered to be a pair, and multiple pair of electrodes are formed as the multi-pair configuration, and they are connected in series.
(4) A configuration is that the width and thickness of the sensing electrode 3 and the reference electrode 4 are arbitrarily selected.

Although in the embodiments, NASICON has been used as the solid electrolyte substrate 2, β alumina, lanthanum fluoride (LaF₃) and the like that have ion conductivity at a temperature zone near room temperature may be used as the solid electrolyte substrate 2 of the present invention.

Although in the embodiments, Bi₂O₃ has been used as the metal oxide to be added to the electrode material Pt of the sensing electrode 3, any metal oxide, if the metal oxide has oxidation activity level such that CO can be oxidized to CO₂ (CO₂ gas), is applicable to the metal oxide of the present invention.

Therefore, as the metal oxide, vanadium oxide (V₂O₅), tungsten oxide (WO₃), molybdenum oxide (MoO₃), cobalt oxide (CoO), manganese oxide (Mn₂O₃), ferric oxide (Fe₂O₃), chrome oxide (Cr₂O₃), nickel oxide (NiO), tin oxide (SnO₂), rhodium oxide (Rh₂O₃), iridium oxide (IrO₂), ruthenium oxide (RuO₂), silver oxide (AgO) and the like may be used.

(Application Example)

It is obvious that since the CO sensor according to the present invention is possible to be down-sized, it can be embedded as the CO sensor device and applied to a portable or stationary CO gas concentration meter, a CO gas sensing alarm apparatus and the like, which are used in a work site indoor/outdoor or adjacent to a gas apparatus indoor.

### INDUSTRIAL APPLICABILITY

The CO sensor according to the present invention can be applied to various types of sensing apparatus, concentration measurement instrument and the like that sense CO gas.

### DESCRIPTION OF CODES

1: CO sensor
2: solid electrolyte substrate
3: sensing electrode
4: reference electrode
5: wire
6: wire

1. A CO sensor characterized in that
a sensing electrode and a reference electrode are mounted on a solid electrolyte substrate;
an ion conductive material having ion conductivity at a temperature zone near room temperature is used for the solid electrolyte substrate;
Pt is used for the reference electrode; and
a calcined compact of Pt, to which metal oxide is added, is used for the sensing electrode having a gas sensing function and a charge collecting function, wherein the metal oxide has oxidation activity to oxidize CO to CO₂.

2. The CO sensor of claim 1 characterized in that
NASICON is used for the solid electrolyte substrate; and
the metal oxide added to the sensing electrode is Bi₂O₃.

2. The CO sensor of claim 2 characterized in that an additive amount of the Bi₂O₃ is 0.1 mass % or more and preferably 1 mass % or more but 30 mass % or less.

4. In a CO sensor comprising a solid electrolyte substrate which includes an ion conductive material having ion conductivity at a temperature zone near room temperature, a sensing electrode being mounted on the solid electrolyte substrate, the sensing electrode having a gas sensing function and a charge collecting function, the sensing including metal oxide which has oxidation activity to oxidize CO to CO₂, and a reference electrode mounted on the solid electrolyte substrate,
a method of manufacturing the CO sensor, comprising the steps of:
using Pt for the reference electrode;
using a mixed paste for the sensing electrode, the mixed paste being formed by mixing metal oxide with a Pt paste;
printing the mixed paste on the solid electrolyte substrate;
printing the Pt paste thereon; and
forming the sensing electrode by calcinating at a predetermined temperature.
